(19) United States
(12) Patent Application Publication
(10) Pub. No.: US 2022/0274774 A1
(43) Pub. Date: Sep. 1, 2022

(54) ENERGY-SAVING AND ENVIRONMENT-FRIENDLY GARBAGE DISPOSAL BOX CAPABLE OF DISPERSING GARBAGE

(71) Applicant: Zhenkun Wang, Marblehead, MA (US)

(72) Inventor: Zhenkun Wang, Marblehead, MA (US)

(21) Appl. No.: 17/238,532

(22) Filed: Apr. 23, 2021

(30) Foreign Application Priority Data
Feb. 26, 2021 (CN) 202120435450.9

(51) Int. Cl. B65F 1/14; B09B 3/00; C12M 1/107; B03B 9/06; B02C 18/00

(52) U.S. Cl. CPC B65F 1/1405; B09B 3/0083; C12M 21/04; B03B 9/06; B02C 18/0084; B65F 2001/1489

(57) ABSTRACT

An energy-saving and environment-friendly garbage disposal box capable of dispersing garbage, applicable to various areas, includes a domestic garbage disposal unit, a recyclable garbage disposal unit and a plastic garbage disposal unit. A pedal mashing device is provided to a rear portion of the domestic garbage disposal unit. The garbage disposal box is covered by solar panels and a computer display. An auto electronic weighing plate is provided to top the garbage disposal box. A set of mashing cutters is disposed inside the garbage disposal box to connect spatially with a fermentation area, and an organic fertilizer area is located under the fermentation area. The recyclable garbage disposal unit is furnished with a garbage entrance at an upper portion thereof and three external drawers corresponding to three recycle chambers. The plastic garbage disposal unit covered by solar panels is furnished thereinside a gravity compression iron and a battery set. The garbage disposal box provided by this patent disclosure and featured in flexible garbage dispersing functions can resolve environmental degradation caused by garbage piling and environmental pollution caused by the current garbage disposal means, and can also provide a possibility to maximize the garbage utilization rate.

### CROSS REFERENCE TO RELATED APPLICATION

This application claims the benefits of China application Serial No. 202120435450.9, filed on Feb. 26, 2021, the disclosures of which are incorporated by references herein in its entirety.

### TECHNICAL FIELD

The present disclosure relates in general to an energy-saving and environment-friendly garbage disposal box capable of dispersing garbage, which is applicable to a variety of areas, such as rural residential areas, urban residential communities and dormitory areas, and which belongs to the field of new types of garbage collecting tools.

### BACKGROUND

Currently, methods of garbage disposal in the world include basically burying and burning. The burying method would occupy tremendous land resources. The burning method would also occupy lots of land resources for piling up the collected garbage prior to the burning process. Gradually, it is highly possible that a situation of city besieged by the collected garbage would be foreseen. In addition, garbage collecting requires a large amount of transportation costs, also an adverse effect upon the environment for transporting the collected garbage is definitely inevitable, and from which a vicious circle in handling the municipal garbage would be formed no matter what the burning or burying method us used. Moreover, the diesel used for burning the garbage would form a waste in energy, and the resulted carbon emissions would cause secondary pollution to the environment and also endanger human health.

This disclosure is related to an energy-saving and environment-friendly garbage disposal box capable of dispersing garbage that provides answers for the aforesaid problems. The energy-saving and environment-friendly garbage disposal box capable of dispersing garbage provided in his disclosure would be applicable to various areas such as rural residential areas with village groups as the unit, urban residential communities or dormitory areas distributed in residential areas in the form of buildings. The method adopted in this disclosure is to recycle and dispose the garbage in a decentralized manner, not a centralized treatment. Thereupon, garbage disposal companies can separate, compress and reuse the plastic and recyclable garbage in a more convenient and efficient way. Thus, the degradable domestic waste can be scattered and treated in situ, and further converted into usable fertilizers for public greening or for growing flowers and vegetables. As such, the transportation cost can be substantially saved. The energy-saving and environment-friendly garbage disposal box capable of dispersing garbage of this disclosure is provided without special area placement requirements, needs no wire and no pipe, has no peculiar smell and a beautiful appearance, and is easy to carry and install. While being installed in a garden, close to a big tree, or roadsides, a biogas lamp of this garbage disposal box can also provide lighting.

The garbage box involved in the this disclosure provides targeted solutions for different types of garbage, so that the garbage can be turned into treasure, and the availability of the garbage can be tapped to the greatest extent.

### SUMMARY

An object of the present disclosure is to solve the current environmental pollution caused by garbage siege, the land occupation by a large amount of non-degradable garbage, the waste of energy caused by the use of diesel incineration of garbage, the carbon emissions causing secondary pollution to the environment, and the resulted ecological issues associated any of above. Installation of the energy-saving and environment-friendly garbage disposal box capable of dispersing garbage provided in this disclosure is scientific, concise, reasonable, beautiful and low cost. The garbage disposal box of this disclosure can disperse garbage in situ, with a high garbage recovery rate and a wide range of applications. It can be used in rural areas, towns, and especially large cities where land resources are in short supply. In particular, extensive use of plastic packaging, whose material cannot be degraded in the soil, has directly endangered the living environment of humans. If the plastic packing material is swallowed by an animal, especially one of aquatic animals, it can't be excreted easily. However, in the current stage, the use of the plastic bags can't be completely gotten rid of. In the energy-saving and environment-friendly garbage disposal box capable of dispersing garbage of this disclosure, solar energy is introduced to heat a metal plate so as to further soften the plastics, instead of burning the plastics to pollute the atmosphere. In addition, the weight of the metal plate would squeeze the softened plastics to reduce the volume thereof. Thereupon, recycling of the plastics would become much easier, and the environmental and ecologic influence from arbitrary disposing the plastic products would be substantially reduced.

In order to achieve the aforesaid objects, following technical solutions are adopted in this disclosure.

In this disclosure, an energy-saving and environment-friendly garbage disposal box capable of dispersing garbage, applicable to various fields, adopts a completely different concept from the previous forms of garbage recycling by transforming the original method of centralized garbage recycling into a new method featured in: (1) collecting according to different types of garbage; (2) dispersing in situ and processing recyclable waste in a real-time manner; and, (3) introducing solar energy to speed up the disposal of garbage, especially upon the degradable domestic garbage, so as to improve the living environment.

In one embodiment of this disclosure, the energy-saving and environment-friendly garbage disposal box capable of dispersing garbage includes a domestic garbage disposal unit (see FIG. 1 Label 1), a recyclable garbage disposal unit (see FIG. 1 Label 2), and a plastic garbage disposal unit (see FIG. 1 Label 3). Through these different units to correctly assort and manage the garbage, convenience, flexibility and effective targeting upon garbage disposal can be enhanced. In this garbage disposal box, three sections in appearance are included. One lateral section of the garbage disposal box is the domestic garbage disposal unit, and another opposite lateral section thereof is the plastic garbage disposal unit. A middle section between the aforesaid two lateral sections is further separated into three isolated parts, orderly from right to left: a paper garbage recycling unit (see FIG. 1 Label 11), a scrap metal garbage recycling unit (see FIG. 1 Label 12) and a battery-chemical contamination garbage recycling unit (see FIG. 1 Label 13). The entire garbage disposal box are furnished with two hand cranks (see FIG. 1 Label 8, 10), one to each lateral side. A biogas lamp (see FIG. 1 Label 7) is provided to a top of the garbage disposal box. A human-machine information exchange electronic display (see FIG. 1 Label 6) and a human-machine information exchange keyboard (see FIG. 1 Label 5) are provided to an upper front portion of the domestic garbage disposal unit, while a rear portion thereof is a pedal mashing device (see FIG. 2 Label 20).

An application of this energy-saving and environment-friendly garbage disposal box capable of dispersing garbage can be following. (1) While in the very first use, registration is required for acquiring a permission to use the instant garbage disposal box in this area, by entering household information via the human-machine information exchange keyboard and further binding a bank card. (2) After entering the user name or scanning the QR code with your mobile phone, a lid or cover on the top of the garbage disposal box can then be opened. (3) If the domestic garbage including a detectable metal or oversized unbreakable garbage is placed on the weight-calculating plate, the display will advise to remove the metal and the oversized unbreakable garbage. After necessary removal, the garbage will be weighed, and the display will show the corresponding fee for crushing this garbage. Then, two options are provided at this time. (1) One option is to pay the fee, by inserting coins via a coin slot (see FIG. 1 Label A) or inserting a bank card into a card slot (see FIG. 1 Label B) to transfer the required fee. As soon as the payment of the fee is confirmed, the weight-calculating plate would be actuated to open so as to drop the garbage thereon into a garbage filter. Simultaneously, the display would show the fee has been paid, and options of the hand crank and the pedal mashing device are provided to be determined for processing the garbage. In this stage, the hand crank or the pedal mashing device can be chosen via clicking the display to crush the garbage, and a receiving account shall be inputted at the same time. After the garbage is processed, the display will show a corresponding amount to be automatically transferred into the receiving account. Thereupon, both exercise and additional income can be obtained. Namely, through utilizing the garbage disposal box of this disclosure, a time for people to exercise and a new channel for people to make money are provided simultaneously. (2) Another option is to apply the hand crank or the pedal mashing device to crush the garbage. This option requires no fee, and the weight-calculating plate would be opened automatically to drop the garbage into the garbage filter. The display will advise that a crush operation can begin, and then the hand crank or the pedal mashing device can be applied to crush the garbage. After the operation of the hand crank or the pedal mashing device, a “thanks” message would be displayed on the display. Thereupon, the charge for processing the garbage can be saved, and the exercise benefit can be obtained as well.

Further, the domestic garbage disposal unit can be covered by solar panels. The human-machine information exchange electronic display (see FIG. 1 Label 6) is disposed at an upper potion in the front side of the garbage disposal box, and the pedal mashing device (see FIG. 2 Label 20) is disposed to the back side (i.e., rear side) of the garbage disposal box. The pedal mashing device is connected with a power transmission chain (see FIG. 2 Label 19) which is furnished with a worm gear shaft (see FIG. 2 Label 18).

Further, a rain entrance hole (see FIG. 3 Label 23) is provided to a top of the domestic garbage disposal unit. The rain water would enter a rain receiving pipe (see FIG. 5 Label 28) in communication with a fermentation area (see FIG. 5 Label 43) inside the garbage disposal box. At a lower-rear portion of the garbage disposal box, a drainage (see FIG. 5 Label 21) is provided thereto. The hand crank (see FIG. 5 Label 8) is installed to a right side of the garbage disposal box for processing the domestic garbage. Under the hand craft for processing the domestic garbage, an organic fertilizer exit (see FIG. 4 Label 27) is provided to further include a machine insertion port for recycling organic fertilizer (see FIG. 4 Label C), such that the resulted organic fertilizer from the domestic garbage can be periodically collected by applying a vacuum device to connect the machine insertion port.

Further, the domestic garbage disposal unit is furnished with an external biogas lamp (see FIG. 1 Label 7) in communication with a biogas pipe (see FIG. 5 Label 29) and further the fermentation area (see FIG. 5 Label 43) inside the garbage disposal box. In addition, heating wires (see FIG. 8 Label 41) are provided to an inner wall of the domestic garbage disposal unit.

Further, a garbage filter (see FIG. 8 Label 40) is disposed inside the domestic garage disposal unit, and the garbage filter further includes thereinside a power transmission bar (see FIG. 8 Label 39). The power transmission bar is furnished with two small mashing cutters (see FIG. 9 Label 44) and a large mashing cutter (see FIG. 9 Label 45).

Further, the fermentation area (see FIG. 8 Label 42) is located below the garbage filter inside the domestic garbage disposal unit. An organic fertilizer storage area (see FIG. 8 Label 43) is provided below the fermentation area, and a battery (see FIG. 8 Label 31) for heating wire and a battery (see FIG. 8 Label 31) for human-machine information exchange electronic display and keyboard are provided below the organic fertilizer storage area.

Further, a battery-chemical contamination recycling unit/bin (see FIG. 1 Label 13), a scrap metal garbage recycling unit/bin (see FIG. 1 Label 12) and a paper garbage recycling unit/bin (see FIG. 1 Label 11) are provided to the top of the recyclable garbage disposal unit.

Further, in front side of the recyclable garbage disposal unit, a paper garbage collecting entrance (see FIG. 3 Label 24), a scrap metal garbage collecting entrance (see FIG. 3 Label 25) and a battery-chemical contamination collecting entrance (see FIG. 3 Label 26) are provided thereto.

Further, the garbage disposal box is further furnished thereinside a battery-chemical contamination garbage chamber (see FIG. 5 Label 34), a scrap metal garbage chamber (see FIG. 5 Label 33) and a paper garbage chamber (see FIG. 5 Label 32).

Further, the plastic garbage disposal unit is furnished thereinside with a power generation plate (see FIG. 5 Label 36), and a battery (see FIG. 5 Label 37) for the power generation plate is located therebelow.

In comparison with the prior art, the garbage disposal box provided in this disclosure can disperse the garbage in situ, and maximize its usability. To different types of garbage, the garbage disposal box makes possible an effective recycling and disposing means in a real-time manner. In addition, the domestic garbage can be transformed into organic fertilizer to be directly utilized for greening, flowering and vegetabling in the surrounding communities. Thereupon, the ecological environment can be further protected, and specific types of garbage to be recycled can be precise. Further, with multiple garbage entrances to the top of the garbage disposal box and garbage exits to the lower portion thereof, the garbage disposal through the garbage disposal box of the disclosure can be much user-friendly and more convenient. Also, with thermally compressing the plastic garbage, the following process of collecting the garbage can be much easier.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given herein below and the accompanying drawings which are given by way of illustration only, and thus are not limitative of the present disclosure and wherein:

FIG. 1 is a schematic front-side perspective view of an energy-saving and environment-friendly garbage disposal box capable of dispersing garbage in accordance with this disclosure;

FIG. 2 is a schematic rear-side perspective view of FIG. 1;

FIG. 3 is a schematic top-side perspective view of FIG. 1;

FIG. 4 is a schematic right-rear-side perspective view of FIG. 1;

FIG. 5 demonstrates schematically interior structures of the energy-saving and environment-friendly garbage disposal box capable of dispersing garbage in accordance with this disclosure;

FIG. 6 demonstrates schematically that the movable heating metal plate is moved upward to top for receiving plastic garbage in the energy-saving and environment-friendly garbage disposal box capable of dispersing garbage in accordance with this disclosure;

FIG. 7 demonstrates schematically that the movable heating metal plate is moved downward to squeeze the plastic garbage in the energy-saving and environment-friendly garbage disposal box capable of dispersing garbage in accordance with this disclosure;

FIG. 8 demonstrates schematically interior structures of the domestic garbage disposal unit of the energy-saving and environment-friendly garbage disposal box capable of dispersing garbage in accordance with this disclosure;

FIG. 9 demonstrates schematically the power transmission bar for the garbage filter of the domestic garbage disposal unit in the Energy-saving and environment-friendly garbage disposal box capable of dispersing garbage in accordance with this disclosure; and

FIG. 10 is a schematic right-side perspective view of FIG. 1.

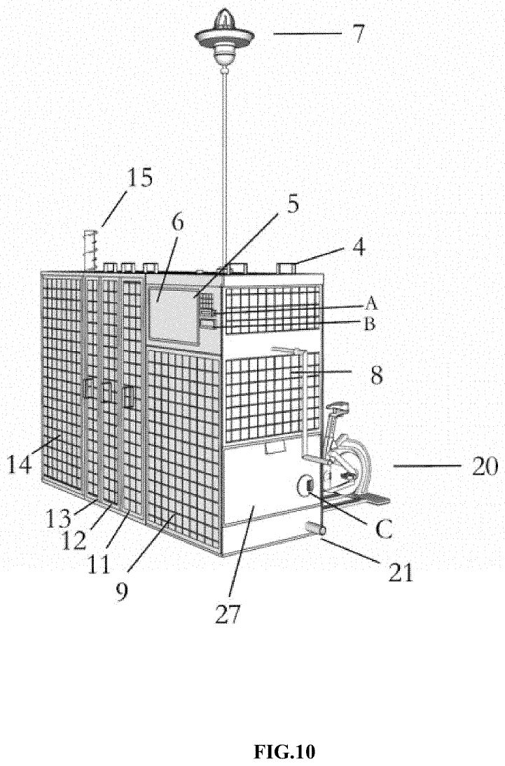

### DESCRIPTIONS FOR LABELS

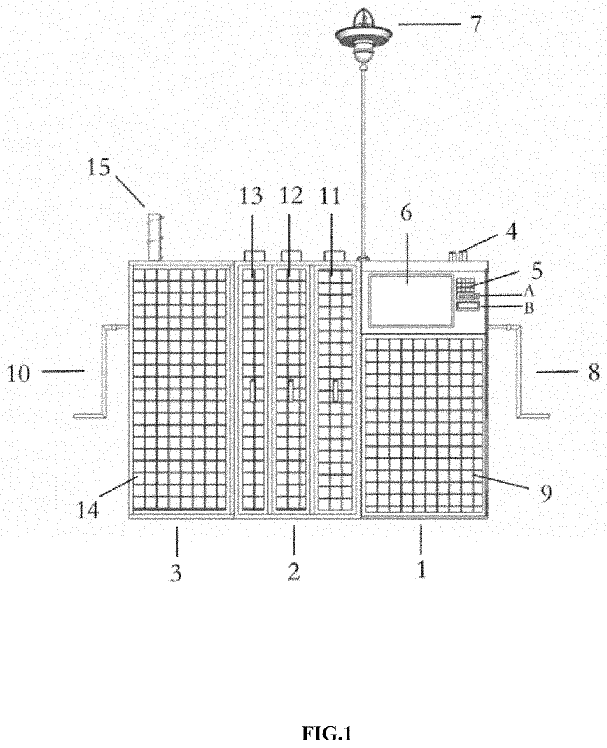

FIG. 1: 1, Domestic garbage disposal unit; 2, Recyclable garbage disposal unit; 3, Plastic garbage disposal unit; 4, Domestic garbage entrance; 5, Human-machine information exchange keyboard; 6, Human-machine information exchange electronic display; 7, Biogas lamp; 8, Hand crank of domestic garbage disposal unit; 9, First front solar panel of domestic garbage disposal unit; 10, Hand crank of heating-squeeze plate for plastic garbage disposal unit; 11, Paper garbage recycling unit/bin; 12, Scrap metal garbage recycling unit/bin; 13, Battery-chemical contamination garbage recycling unit/bin; 14, Front solar panel of plastic garbage disposal unit; 15, Spiral rotating bar; A, Coin slot; B, Card slot.

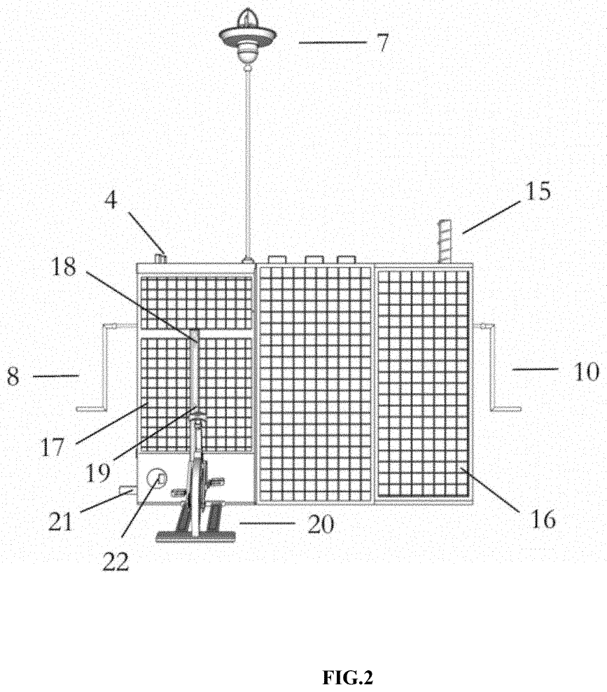

FIG. 2: 16, Rear solar panel of plastic garbage disposal unit; 17, Rear solar panel of domestic garbage disposal unit; 18, Worm gear shaft; 19, Power transmission chain; 20, Pedal mashing device; 21, Drainage; 22, Baking powder hole.

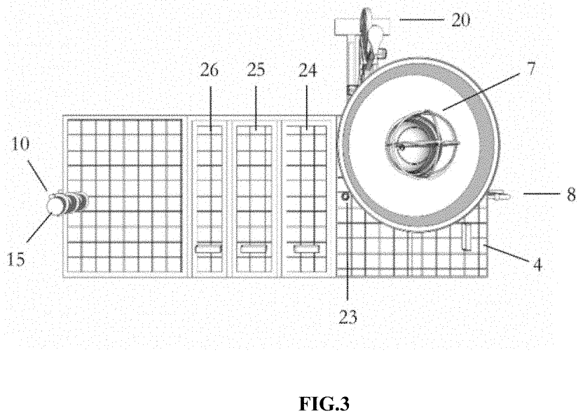

FIG. 3: 23, Rain entrance hole; 24, Paper garbage collecting entrance; 25, Scrap metal garbage collecting entrance; 26, Battery-chemical contamination collecting entrance.

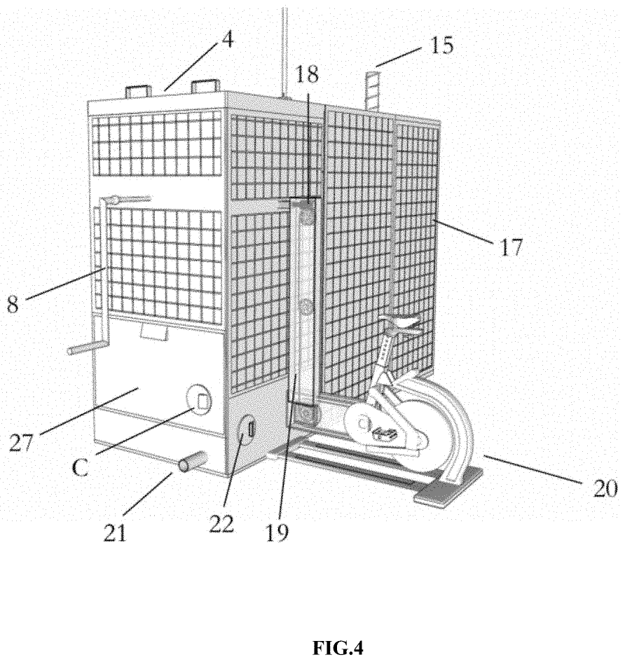

FIG. 4: 27, Organic fertilizer exit; C, Machine insertion port for recycling organic fertilizer.

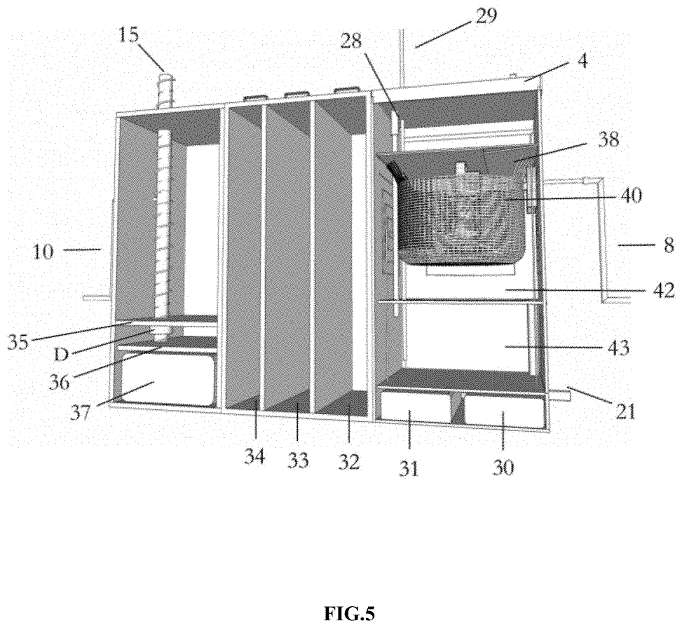

FIG. 5: 28, rain receiving pipe; 29, Biogas pipe; 30, Battery for heating wire; 31, Battery for human-machine information exchange electronic display and keyboard; 32, Paper garbage chamber; 33, Scrap metal garbage chamber; 34, Battery-chemical contamination garbage chamber; 35, Movable heating metal plate; 36, Power generation board; 37, Battery for heating metal plate; D, Bearing of heating-squeeze plate.

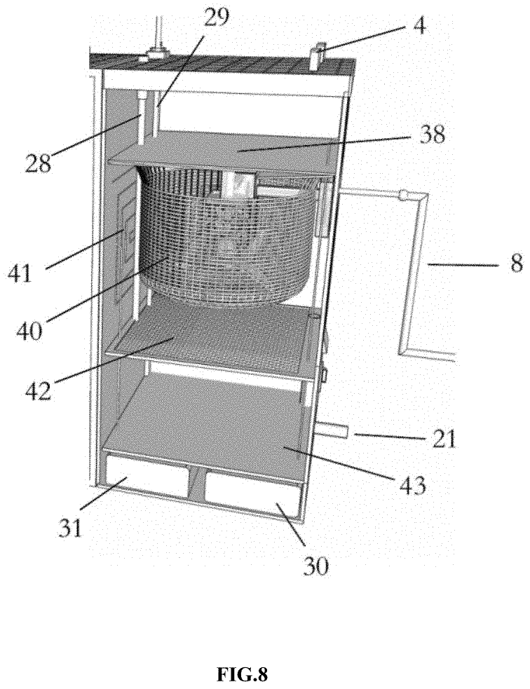

FIG. 8: 38, Weight-calculating plate; 40, Garbage filter; 41, Heating wire; 42, Fermentation area; 43, Organic fertilizer storage area.

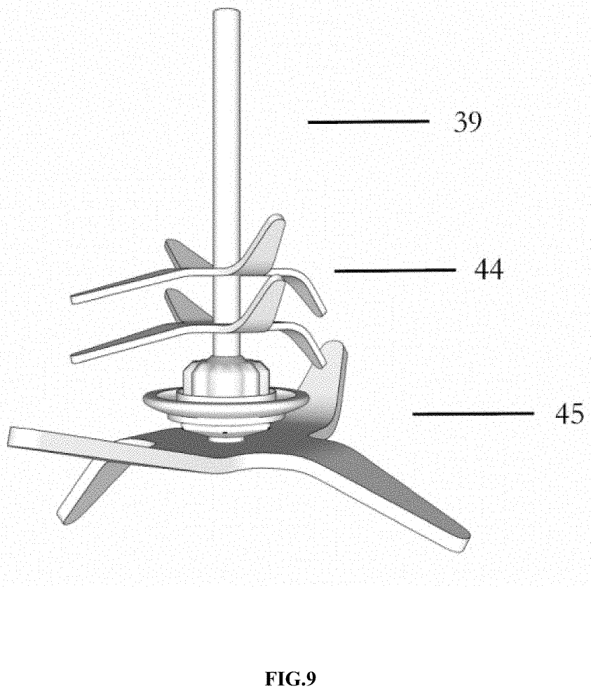

FIG. 9: 39, Power transmission bar; 44, Small mashing cutter; 45, Large mashing cutter.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

Referring to FIG. 1 and FIG. 2, an energy-saving and environment-friendly garbage disposal box capable of dispersing garbage includes a domestic garbage disposal unit 1, a recyclable garbage disposal unit 2 and a plastic garbage disposal unit 3. As shown in FIG. 1 and FIG. 8, the domestic garbage disposal unit 1 is furnished externally with a first front solar panel 9 and a rear solar panel 1, and both of which cover the box structure so as to obtain the energy-saving and environment-friendly solar energy 16 for promoting a garbage-recycling process in the domestic garbage disposal unit 1. A human-machine information exchange keyboard 5 and a human-machine information exchange electronic display 6 are located exteriorly at an upper portion in the front side of the domestic garbage disposal unit 1 for interacting with the user and for concisely displaying information. When a user confirms a message shown on the human-machine information exchange electronic display 6, the hand crank 8 of the domestic garbage disposal unit 1 can be used to open the domestic garbage entrance 4, and then the internal weight-calculating plate 38 can be utilized to determine the garbage weight and to judge whether or not the garbage has been correctly assorted. As it is confirmed that the domestic garbage has been dropped into the garbage filter 40 inside the garbage disposal box, then the pedal mashing device 20 located behind the domestic garbage disposal unit 1 can be applied to rotate the worm gear shaft 18 through the power transmission chain 19, and to further rotate the power transmission bar 39 inside the domestic garbage disposal unit 1. Thus, the domestic garbage in the garbage filter 40 would be cut and mashed by two small mashing cutters 44 and a large mashing cutter 45 of the power transmission bar 39. The mashed garbage would then pass the garbage filter 40 and enter the fermentation area 42. In addition, baking powders can be added into the domestic garbage disposal unit 1 through the baking powder hole 22, and rain water would be also led to the fermentation area 42 inside the domestic garbage disposal unit 1 via the rain receiving pipe 28. The heating wire 41 on the inner wall of the domestic garbage disposal unit 1 would raise the internal temperature ready for the following fermentation. When a specific amount of biogas is accumulated in the fermentation area, the biogas would pass through the biogas pipe 29 to light up the biogas lamp 7 on top of the domestic garbage disposal unit 1. After being fully fermented, the domestic garbage inside the domestic garbage disposal unit 1 would be dropped into the organic fertilizer storage area 43 for related personals to remove away for secondary usage. At the lower portion of the domestic garbage disposal unit 1, the battery 30 for heating wire and the battery for human-machine information exchange electronic display and keyboard 31 are disposed separately. The battery 30 for heating wire is connected electrically with the heating wire 41 on the inner wall of the garbage disposal box to provide thermal protection for the entire domestic garbage disposal unit 1.

As shown in FIGS. 1-3 and FIG. 5, the recyclable garbage disposal unit 2 is furnished externally, at an upper portion thereof with three garbage collecting entrances; i.e., a paper garbage collecting entrance 24, a scrap metal garbage collecting entrance 25 and a battery garbage collecting entrance 26, located correspondingly to the paper garbage chamber 32, the scrap metal garbage chamber 33 and the battery-chemical contamination garbage chamber 34 inside the recyclable garbage disposal unit 2, respectively. When any type of garbage is accumulated to a specific amount, the related personal would carry out the recycling through the paper garbage recycling unit/bin 11, the scrap metal garbage recycling unit/bin 12 and the battery-chemical contamination recycling unit/bin 13 on the recyclable garbage disposal unit 2. In the recyclable garbage disposal unit 2, the garbage for recycling is strictly assorted. With the three separated collecting entrances for three recycling units/bins, the related personal can proceed the recycling more conveniently and more correctly, and from which the following secondary garbage recycling can be much benefited.

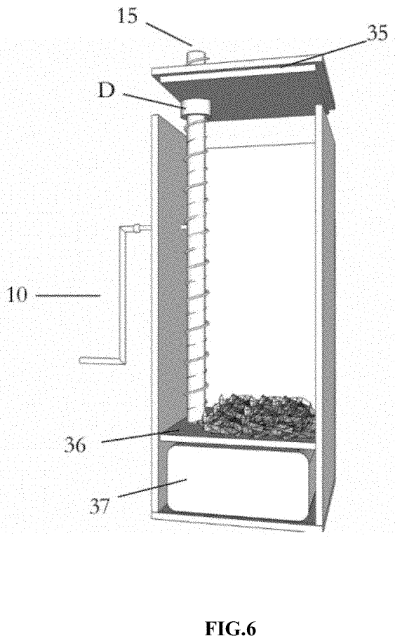

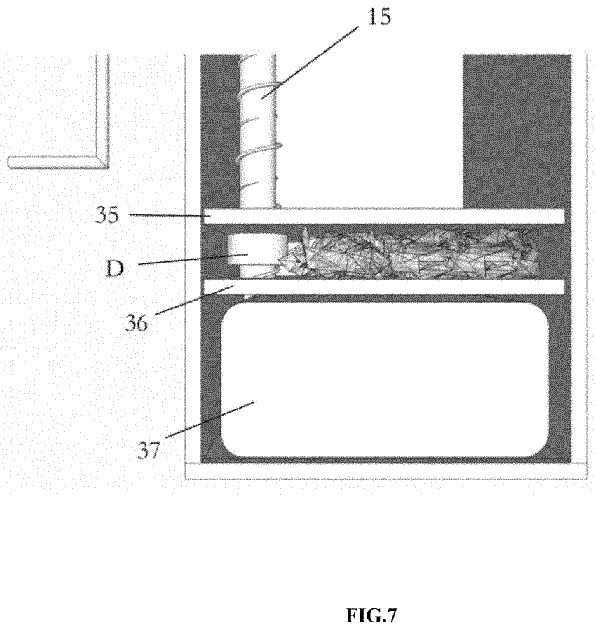

As shown in FIG. 1, FIG. 5, FIG. 6 and FIG. 7, the plastic garbage disposal unit 3 is furnished externally with a front solar panel 14 and a rear solar panel 16, and both of which cover the garbage disposal box to a maximum extent, and are applied to provide electricity for supporting the plastic garbage disposal unit 3 to perform better recycling of the plastic garbage. While the hand crank 10 of the heating-squeeze plate for the plastic garbage disposal unit 10 is rotated, the bearing D would be driven to move up and down with the movable heating metal plate 35. When the movable heating metal plate 35 is moved upward to reach the external entrance of the plastic garbage disposal unit 3, if plastic garbage is further placed into the plastic garbage disposal unit 3, then the movable heating metal plate 35 would be lowered automatically with the baring D so as to compress the plastic garbage onto the power generation plate 36. Then, the movable heating metal plate 35 and the power generation plate 36 would begin to heat up and soften the plastic garbage, and simultaneously the plastic garbage would be squeezed between the movable heating metal plate 35 and the power generation plate 36. Hence, the plastic garbage is softened and also compressed into a minimum reduced volume at this current stage. When the plastic garbage is softened and compressed into a minimum volume, the corresponding recycling of the plastic garbage would be much easier, and also the adverse influence upon the ecological environment by the plastic garbage would be substantially reduced.

With respect to the above description then, it is to be realized that the optimum dimensional relationships for the parts of the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

What is claimed is:

1. A disposal box, comprising:
a first garbage disposal unit for a household garbage, comprising:
a bio gas pipe; and
a garbage filter for collecting the household garbage; and
a biogas lamp, where a bio gas is generated from the household garbage and exits via the bio gas pipe to light up the biogas lamp.

2. The disposal box as claimed in claim 1, further comprising:
a rain receiving pipe;
a drainage; and
a baking powder hole for inputting a baking powder.

3. A disposal box, comprising:
a solar panel;
a first garbage disposal unit for a household garbage, comprising:
a pedal mashing device;
a chain;
a worm gear shaft; and
a mashing cutter;
wherein the pedal mashing device drives the mashing cutter via the chain and the worm gear shaft to smash the house hold garbage.

4. The disposal box as claimed in claim 3, wherein the pedal mashing device is a stationary bicycle.

5. A disposal box, comprising:
a human-machine information exchange electronic display;
a solar panel;
a first garbage disposal unit for a plastic garbage, comprising:
a first plate;
a second plate;
a spiral rotating bar; and
a power source;
wherein the plastic garbage is placed between the first plate and the second plate, and the first plate is heated and pressed down on the plastic garage via the spiral rotating bar.

6. The disposal box as claimed in claim 5, further comprising:
a hand crank for moving the first plate along the spiral rotating bar.

7. The disposal box as claimed in claim 5, further comprising:
a second garbage disposal unit for recycling, comprising:
a paper garbage chamber for collecting a paper garbage;
a scrap metal garbage chamber for collecting a scrap metal; and
a battery-chemical contamination garbage chamber for collecting a battery-chemical contamination garbage; and
a third garbage.
